(19) United States
(12) Patent Application Publication
(10) Pub. No.: US 2024/0050266 A1
(43) Pub. Date: Feb. 15, 2024

(54) POWERED INTRAORAL APPARATUS

(71) Applicant: Gwo-Tzong Hwang, Bethesda, MD (US)

(72) Inventor: Gwo-Tzong Hwang, Bethesda, MD (US)

(21) Appl. No.: 17/885,529

(22) Filed: Aug. 10, 2022

(51) Int. Cl. A61F 5/56; H04R 1/08

(52) U.S. Cl. CPC A61F 5/566; H04R 1/08; H04R 2460/13; A61F 2005/563; H04R 2420/07

(57) ABSTRACT

An approach is disclosed for transmitting customizable audio signals through teeth activated by pressure. An intraoral apparatus is provided with a pressure switch controlled by receiving pressure. The pressure switch holds a state where the state is one of on and off. The intraoral apparatus includes a power source, an audio element, a control element, and a bone conduction element. A customizable audio signals is received by the audio element. When pressure is received by the pressure switch when the state is off, the control element can be charged from the power source with a charge. The charge enables sending the customizable audio signal through the bone conduction element. In some configuration the charge is needed, as without the charge the customizable audio signals is not transmitted.

### BACKGROUND

The present invention relates to the field of intraoral apparatus, more specifically to deliver customizable audio signals for a limited time to a user through teeth.

### SUMMARY

According to one embodiment of the invention, there is provided a method for sending customizable audio signals through teeth activated by pressure. An intraoral apparatus is provided with a pressure switch controlled by receiving pressure. The pressure switch holds a state where the state is one of on and off. The intraoral apparatus includes a power source, an audio element, a control element, and a bone conduction element. Customizable audio signals are received by the audio element. When pressure is received by the pressure switch when the state is off, in some configurations the control element is charged from the power source with a charge. In some configurations the charge enables sending the customizable audio signals through the bone conduction element, and without the charge the customizable audio signals cannot be sent.

According to one embodiment of the invention, there is an intraoral apparatus configured to send customizable audio signals through teeth activated by pressure. The intraoral apparatus includes a pressure switch controlled by receiving pressure that holds a state where the state is one of on and off, a power supply (power source), an audio element, a control element, and a bone conduction element. The audio element receives customizable audio signals. When the state is off and pressure is received by the pressure switch, in some configurations the control element is charged from the power source with a charge. In some configurations the charge enables sending the customizable audio signals through the bone conduction element, and without the charge the customizable audio signals cannot be sent.

The foregoing is a summary and thus contains, by necessity, simplifications, generalizations, and omissions of detail; consequently, those skilled in the art will appreciate that the summary is illustrative only and is not intended to be in any way limiting. Other aspects, inventive features, and advantages of the present invention will be apparent in the non-limiting detailed description set forth below.

### BRIEF DESCRIPTION OF THE DRAWINGS

The present invention may be better understood, and its numerous objects, features, and advantages made apparent to those skilled in the art by referencing the accompanying drawings, wherein:

FIG. 1 is a schematic operational flow of an embodiment of the invention disclosed herein;

FIG. 2 is a schematic depiction including various components of a first embodiment of an apparatus supporting the operational flow in FIG. 1;

FIG. 3 is a rear-view sketch of the various components of the first embodiment of the apparatus shown in FIG. 2;

FIG. 4 is a right-side view sketch of the various components of the first embodiment of the apparatus shown in FIG. 2;

FIG. 5 is a schematic depiction including various components of a second embodiment of an apparatus supporting the operational flow in FIG. 1; and

FIG. 6 is a schematic depiction including various components of a third embodiment of an apparatus supporting the operational flow in FIG. 1.

### DETAILED DESCRIPTION

The present invention relates to the field of intraoral apparatus configured to treat bruxism. According to National Institutes of Health studies, about eight percent of the general population suffer from nighttime teeth grinding, or sleep bruxism. Statistics from a study of bite force and state of dentition shows the strength of an adult human bite can range around 11 kilogram-force (KGF) to about 39 KGF. Data from a quantitative study of bite force during sleep associated bruxism has shown that the mean amplitude of detected bruxism events was 22.5 KGF and the highest amplitude of nocturnal bite force in individual subjects was 42.3 KGF. The major consequences of sleep bruxism are headaches, tooth wear, and complaints from bed partner due to grinding noises.

Various prior art approaches have been developed to treat bruxism. One type of the prior art includes different shapes of occlusal guards. Different shapes of occlusal guards provide some protection to the teeth from grinding, but they do not stop bruxism. Another type of prior art embodiments uses sensors to monitor bruxism. When bruxism is detected, an audio alarm is triggered to alert/wake up the user to stop the bruxism. However, audio alarms severely disrupt the user and his/her bed partner's sleep and negatively impact their health from lack of sleep. Yet another type of the prior art embodiments also uses sensors to monitor the patient for bruxism, and when the bruxism occurs, electric current, laughing gas, or some medicines are applied to stimulate/relax the oral muscles. These devices might be able to stop the bruxism but are cumbersome and cannot be easily self-administered at home.

In view of the shortcomings in the prior arts, there is an advantage of the disclosed approaches to treat bruxism facilitating self-administered treatment at home. The disclosed approach monitors the user for bruxism. When the bruxism is detected, a bone conduction element, for example, but not limited to, a bone conductor transducer, is used to send some calming music or customizable audio signals preselected by the user. Support may be provided to allow for a comfortable amplification/vibration strength level pre-set by the user, to the user's teeth via occlusal guards. This calming music or audio may be of the user's choice. The music or audio input is received from the bone conduction transducer, which is more likely to interrupt and stop user's bruxism, without waking up the user or his/her bed partner.

The disclosed approach provides an improved apparatus for treating bruxism. An embodiment may have a flexible fastener connecting two housings for the left side and right side of the teeth, each of the two housings may be integrated with an occlusal guard. Each occlusal guard may be integrated with a pressure switch. The housing may be perpendicularly connected to the occlusal guard and sit in an oral vestibule, the area between the teeth and the cheeks. The housing may include a control element, for example, but not limited to, a circuit control element, an audio element, at least one bone conductor transducer, and a power supply (power source.) The circuit control element may further comprise a long bite circuit, a delay off circuit, and an auto off circuit. The apparatus may further include an audio memory element. The power supply may be, for example, but not limited to, a disposable battery, a rechargeable battery that can be recharged from external power sources, etc. In some embodiments, the power supply may use energy derived from the user.

The occlusal guard provides protection to the teeth when bruxism occurs. In an embodiment, a pressure switch is connected to the circuit control element. The pressure switch can be pressed on or pressed off by the user consciously biting and holding down the occlusal guard for longer than some predetermined duration of time (long bite), or by the user's randomly biting down by the teeth grinding from a sleep bruxism (short bite). When the pressure switch is pressed on from an off state, all electronic components, including the circuit control element and the power supply, may be activated. When activated by a short bite, the power supply element fast charges a capacitor in the delay off circuit. When the capacitor is fully charged (either a specific charge up voltage is reached, or when the voltage reaches the thermal noise value of the capacitor, or some other measurement methods), the auto off circuit sets the pressure switch to off state. When the pressure switch is off, the capacitor is not charged. The delay off circuit slowly uses the energy stored in the capacitor to regulate the power supply to continue to provide needed power to all electronic components in the housing until the capacitor is fully drained, or the power supply is fully drained. The pressure switch is likely to be pressed multiple times during a bruxism event, the delay off circuit and auto off circuit ensure that the customizable audio signals are delivered continuously during the bruxism event and will be stopped shortly after the bruxism event stops. When the pressure switch, is pressed and held for a predetermined duration of time (long bite), from an on state, the following occurs: 1) The pressure switch state is set to off. 2) The energy stored in the capacitor of the delay off circuit is fast drained completely. 3) All electronic components in the apparatus are deactivated to prevent the sending of the customizable audio through the teeth. When the pressure switch, is pressed and held for a predetermined duration of time (long bite), from an off state, the following occurs: 1) The pressure switch state is set to on. 2) The power supply is activated to enable the sending of the customizable audio through the teeth. 3) The capacitor of the delay off circuit is not charged.

The audio element, when activated, can receive customizable audio signals in a variety of transmission means. Examples of the customizable audio signals may include, for example, but are not limited to, a mobile phone playing user selected calming music at a preferred volume level, via Bluetooth wireless transmissions, and sends the received customizable audio signals to the bone conductor transducer.

When activated, the bone conductor transducer receives the customizable audio signal from the audio element and sends it via vibration to the user's teeth. To increase the sound quality, the bone conductor transducer can be placed in a sealed chamber immediately adjacent to the occlusal guards and the chamber may be filled with non-electricity-conductive material, for example, but not limited to, non-electricity-conductive silicone gel.

In an embodiment, the disclosed apparatus can further comprise an audio memory element. The audio memory element comprises a memory area, an audio player, and functions to upload and store the preferred customizable audio signals onto the memory area and sends the stored customizable audio signals to the bone conductor transducer. Instead of playing customizable audio signals from the audio element, a user can choose to play the previously uploaded customizable audio signals from the audio memory element using the apparatus by uploading and storing customizable audio signals in the audio memory element. If there are no customizable audio signals stored in the audio memory element, the apparatus will play the customizable audio signals from the audio element.

When sleep bruxism occurs, the introduction of the calming customizable audio signals at a comfortable volume is more likely to stop the bruxism without waking up the user or disturbing other persons in the surrounding area, such as a bed partner.

When a user is jogging or swimming, headphones, airpods, and earbuds may fall off from the user's ears or the user's head. The disclosed intraoral apparatus is securely held inside the user's mouth minimizing the chance of falling off. In addition, by having the intraoral apparatus in the user's mouth interference with the surrounding environment is minimized.

The occlusal guard provides protection to the teeth when bruxism occurs. The pressure switch is connected to the circuit control element. The pressure switch may be pressed on or pressed off responsive to the user's actions, such as, by consciously biting down the occlusal guard (long bite) or by the teeth grinding from a bruxism event (short bite).

In an embodiment, when activated by a short bite, the power supply fast charges a capacitor in the delay off circuit. When the capacitor is fully charged, the auto off circuit sets the pressure switch to off state. There are multiple ways to determine when a capacitor is fully charged, for example, but not limited to, determining when a specific charge-up voltage is reached, determining when the voltage reaches the thermal noise value of the capacitor, etc. When the pressure switch state is off, the capacitor is not charged. The delay off circuit slowly uses the energy stored in the capacitor to regulate the power supply to continue to provide needed power to all electronic components in the housing until the capacitor is fully drained. The pressure switch is likely to be pressed multiple times during a bruxism event, the delay off circuit and the auto off circuit ensure that the customizable audio signals are delivered continuously during the bruxism event and will be stopped after the bruxism event stops. In some embodiments, the customizable audio signals delivery is stopped in a limited time, for example, 5 minutes, after the bruxism event stops.

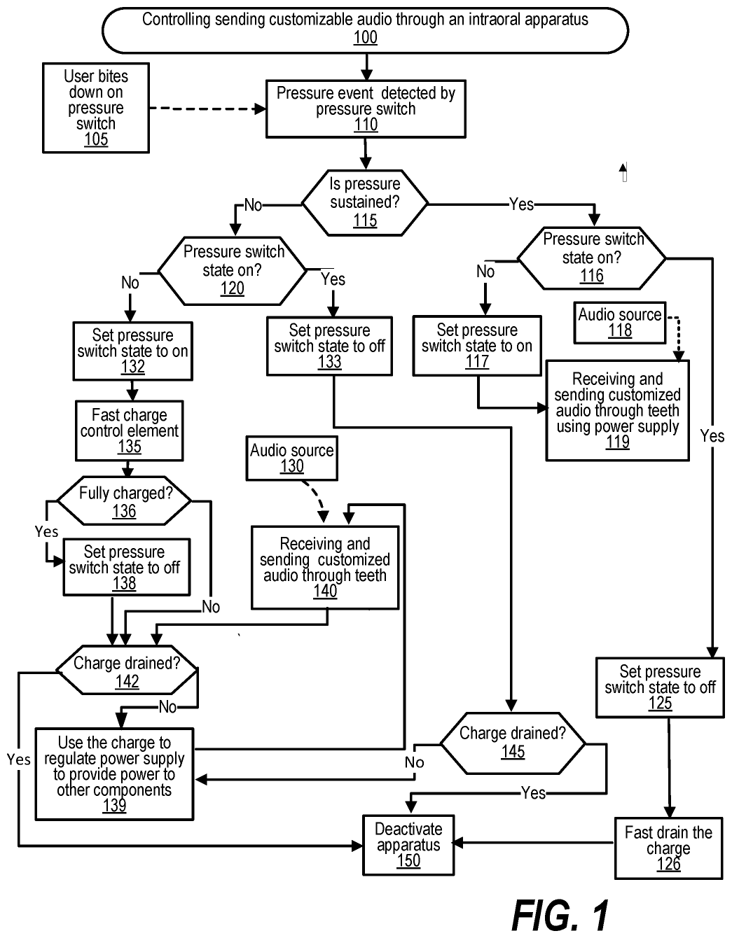

FIG. 1 processing commences at 100 and shows the steps taken by a process that controls sending customizable audio through an intraoral apparatus. At step 105, the user bites down on pressure switch. The biting may be short bites due to grinding teeth, such as, occurs during bruxism. Alternatively, the user may be using long bites to intentionally issue commands that the device will execute based on how the device is configured. In some embodiments, a detection of a pressure exceeding a specific force may cause an action, such as, activating the device or deactivating the device. The amount of time the bite is held may be used to control details of the action. For example, a long bite, say exceeding 10 seconds, may mean immediate deactivation. A short byte, say under 3 seconds, may mean activate the device for a duration of time. At step 110, a pressure event is detected by pressure switch. A pressure event is detected responsive to a user biting down on the intraoral apparatus or grinding teeth. Any bite on the pressure switch may trigger the pressure event detected by the pressure switch 110. If the grip of the teeth on the pressure switch is released and the grinding restarts, a new pressure event may be detected. The process determines as to whether is pressure sustained (decision 115). If pressure is sustained (long bite), then decision 115 branches to the ‘Yes’ branch. If pressure is not sustained (short bite), then decision 115 branches to the ‘No’ branch. When the pressure is first detected, the process may, for example, set a stopwatch used to monitor how long the pressure is sustained. When the pressure switch detects an absence of pressure, the stopwatch is checked to see how long the pressure was maintained. If the stopwatch indicates a predetermined duration of time has elapsed, then decision 115 branches to ‘Yes’ branch (long bite). Alternatively, or additionally, when the pressure is first detected, the process may, for example, set a timer. If the timer triggers an interrupt indicating a set predetermined duration of time has elapsed before the absence of pressure is detected, the process notes that the pressure is sustained and follows the ‘Yes’ branch for decision 115. The process determines if pressure switch state is on (decision 116). If pressure switch state is on, then decision 116 branches to the ‘Yes’ branch. If pressure switch state is not on, then decision 116 branches to the ‘No’ branch. At step 117, the pressure switch state is set to on. At step 119, receiving and sending the customizable audio signals from audio source 118 through teeth using power supply. The customizable audio signals may have been previously downloaded to the audio source 118 or be transferred real-time to the audio source 118. At step 125, the pressure switch state is set to off. At step 126, the process fast drains the charge. At step 150, the intraoral apparatus is deactivated. When decision 115 branches to ‘No’ branch (short bite), the process determines if pressure switch state is on (decision 120). If pressure switch state is on, then decision 120 branches to the ‘Yes’ branch. If pressure switch state is not on, then decision 120 branches to the ‘No’ branch. At step 132, the process sets the switch state to on. At step 133, the process sets the pressure switch state to off. At step 135, the process fast charges control element (fast charging continues when the switch state remains on). The audio source 130 may have been supplied, for example, by downloading an audio file via a wired connection. Alternatively, the audio source 130 may be delivered real-time from a smart phone, via a short-range wireless technology, such as, Bluetooth. The process determines as to whether the control element is fully charged (decision 136). If the control element is fully charged, then decision 136 branches to the ‘Yes’ branch. On the other hand, if control element is not fully charged, then decision 136 branches to the ‘No’ branch. At step 138, the pressure switch state is set to off. The process determines as to whether charge drained (decision 142). If charge drained, then decision 142 branches to the ‘Yes’ branch that goes to step 150, the intraoral apparatus is deactivated. On the other hand, if not charge drained, then decision 142 branches to the ‘No’ branch. The process determines as to whether charge drained (decision 145). If charge is not drained, then decision 145 branches to the ‘No’ branch which goes to 139. At step 139, the process uses the charge from the control element to regulate power supply to provide power to other components. From 139, the process proceeds to step 140, continuing to receive and send customizable audio through teeth which loops back to decision 142. This looping continues until the charge is drained, at which point decision 142 branches to the ‘Yes’ branch exiting the loop.

In addition, the disclosed approach can also be used, more generally, to receive audio signals in a more discreet way from the environment, including, but not limited to, listening to favorite music when jogging, swimming, or studying in library.

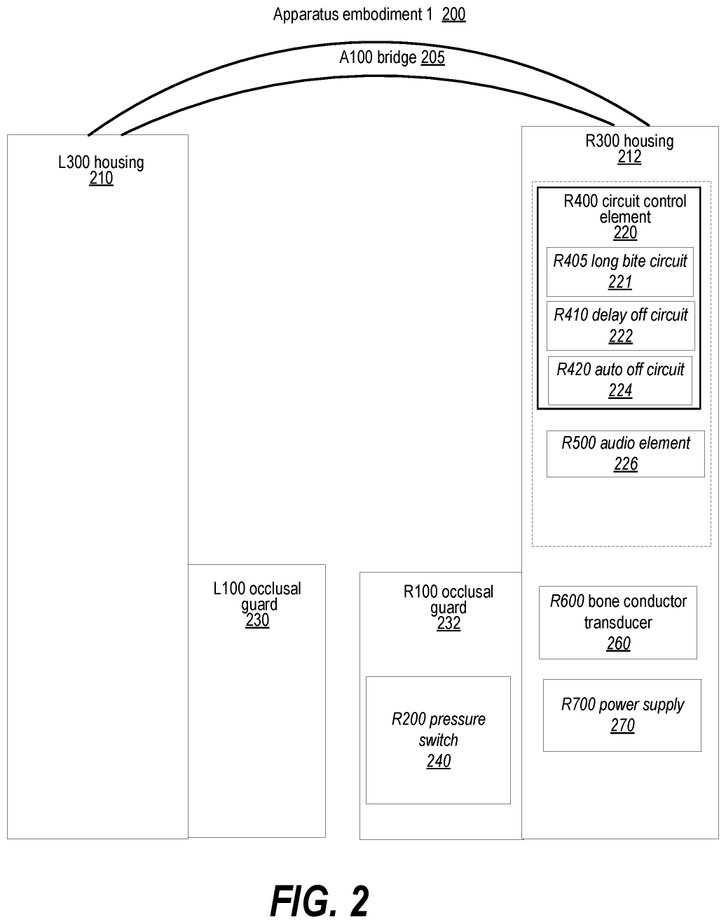

FIG. 2 is a schematic depiction including various components of an apparatus embodiment 1 200 supporting the operational flow in FIG. 1. The apparatus embodiment 1 200 may be shaped like a commonly available basic night guard. An A100 bridge 205 may be a flexible fastener that connects a L300 housing 210 for the left side of a mouth to a R300 housing 212 for the right side of the mouth. These housings may be fitted to be placed in the oral vestibule of the user. Each of the two housings may be integrated with corresponding occlusal guards. The L300 housing 210 is integrated with a L100 occlusal guard 230. The L300 housing 210 sits in the left side of the oral vestibule, that is, the area between the left teeth and the left cheek. The R300 housing 212 is integrated with a R100 occlusal guard 232. The R300 housing 212 sits in the right side of the oral vestibule, that is, the area between the right teeth and the right cheek. A R200 pressure switch 240 is integrated within the R100 occlusal guard 232 and connected to a R400 circuit control element 220 in the R300 housing 212. The R300 housing 212 houses the R400 circuit control element 220, a R500 audio element 226, a R600 bone conductor transducer 260, and a R700 power supply 270. The R400 circuit control element 220 may include a R405 long bite circuit 221, a R410 delay off circuit 222, and a R420 auto off circuit 224. The R600 bone conductor transducer 260 may be placed inside a sealed chamber immediately adjacent to the R100 occlusal guard 232 and filled with non-electricity-conductive silicone gel. The R400 circuit control element 220 has power circuits, data circuits, and process controlling logics that interconnects with and controls all other electronic components in the apparatus embodiment 1 200. The R410 delay off circuit 222 may comprise a capacitor. The R500 audio element 226 may comprise a Bluetooth module.

Before a regular use of the apparatus embodiment 1 200, the user may bite down the R200 pressure switch 240 to activate the apparatus embodiment 1 200, and pair the Bluetooth connection between the Bluetooth module in the audio element 226 of the apparatus embodiment 1 200 and a Bluetooth enabled audio playing device, for example, but not limited to, a mobile phone. The user may bite down the R200 pressure switch 240 again to turn off the apparatus embodiment 1 200 after the Bluetooth between the two devices are paired.

In a scenario for using the apparatus embodiment 1 200 to treat sleep bruxism. The user turns on the previously paired mobile phone with the Bluetooth function enabled, plays continuously his/her favorite calming audio with comfortable volume setting. The mobile phone can be placed in a place where the audio sound cannot be heard by the user, but its location is well within the Bluetooth connecting range.

The user then puts the apparatus embodiment 1 200 in his/her mouth, goes to bed, and falls asleep. If a sleep bruxism occurs and the user grinds his/her teeth, the teeth grinding force will turn the R200 pressure switch 240 state on. Once the switch state is on, the R405 long bite circuit 221 is activated to determine if the R200 pressure switch 240 is pressed and held for a predetermined duration of time or longer (long bite checking). The sleep bruxism normally would not bite down the teeth for a long duration of time, and in most cases, would fail the long bite checking and result as a short bite. When the switch is turned on by the short bite, a capacitor in the R410 delay off circuit 222 is fast charged by the R700 power supply 270.

When the R200 pressure switch 240 is pressed down while its state is on, the R405 long bite circuit 221 is activated to check if the pressed down is a long bite or short bite. A short bite triggers the R400 circuit control element 220 to set the R200 pressure switch 240 state to off and checks if the capacitor in the R410 delay off circuit 222 is fully drained. If the capacitor is fully drained, it deactivates the apparatus embodiment 1 200. If the capacitor is not fully drained, it continues to deliver the customizable audio signals to the user's teeth until the capacitor is fully drained, then it deactivates the apparatus embodiment 1 200. On the other hand, a long bite while the R200 pressure switch 240 state is on triggers the R400 circuit control element 220 to set the R200 pressure switch 240 state to off, fast drain the capacitor in the R410 delay off circuit 222, and deactivates the apparatus embodiment 1 200.

When power is supplied, the Bluetooth module in the R500 audio element 226 will be turned on and automatically pairs with the mobile phone. The mobile phone will automatically reroute the playing audio output to Bluetooth output. The audio received by R500 audio element 226 will then be routed to the R600 bone conductor transducer 260. The R600 bone conductor transducer 260 then delivers the calming customizable audio by vibration means via the R100 occlusal guard 232 to the user.

When the capacitor is fully charged, the R420 auto off circuit 224 sets the R200 pressure switch 240 to the off state. When the R200 pressure switch 240 state is off, the capacitor is not charged. There are multiple ways to determine if the capacitor is fully charged, for example, but not limited to, a specific charge up voltage is reached, or, when the voltage reaches the thermal noise value of the capacitor, etc. The R410 delay off circuit 222 design supports usage of the energy stored in the capacitor to regulate the R700 power supply 270 to continue to provide needed power to all electronic components in the R300 housing until the capacitor is completely drained, or the R700 power supply 270 is fully drained. The R200 pressure switch 240 is likely to be pressed multiple times during a bruxism event and its state will be switched between on and off multiple times. The R410 delay off circuit 222 and the R420 auto off circuit 224 ensure that the customizable audio signals is delivered continuously during the bruxism event and is stopped some short duration of time after the bruxism event stops. The R500 audio element 226, when activated, can receive customizable audio signals via a variety of transmission means. The transmission means include, but not limited to, a mobile phone playing user selected calming music at a preferred volume level, via Bluetooth wireless transmissions, and sends the received the customizable audio signals to the R600 bone conductor transducer 260. When activated, the R600 bone conductor transducer 260 receives the customizable audio signals and sends it via vibration to the user's teeth. To increase the sound quality, the R600 bone conductor transducer 260 can be placed in a sealed chamber immediately adjacent to the R100 occlusal guard 232 and the sealed chamber can be filled with non-electricity-conductive silicone gel.

Users can also use the apparatus when jogging, swimming, or in a library to listen to favorite audio with minimal interference to and from the surrounding environment. In a scenario for using the apparatus embodiment 1 200 when jogging, the user bites and holds down the R200 pressure switch 240 for a longer duration of time, for example, but not limited to, 10 seconds, this activates the R405 long bite circuit 221 to check and confirm that it is a long bite. When the apparatus embodiment 1 200 is turned on by a long bite, the R700 power supply 270 directly provides the power to R400 circuit control element 220, R500 audio element 226, and R600 bone conductor transducer 260. When power is supplied, the Bluetooth module in the R400 circuit control element 220 is turned on and automatically pairs with the mobile phone. The mobile phone automatically reroutes the playing audio output to Bluetooth output. The audio received by R500 audio element 226 is then routed to the R600 bone conductor transducer 260. The R600 bone conductor transducer 260 then delivers the calming customizable audio signals by vibration means via the R100 occlusal guard 232 to the user. Because the capacitor in the R410 delay off circuit 222 is not charged under this scenario, the user can bite down the R200 pressure switch 240 again, either via a long bite or a short bite, to immediately deactivate the apparatus embodiment 1 200.

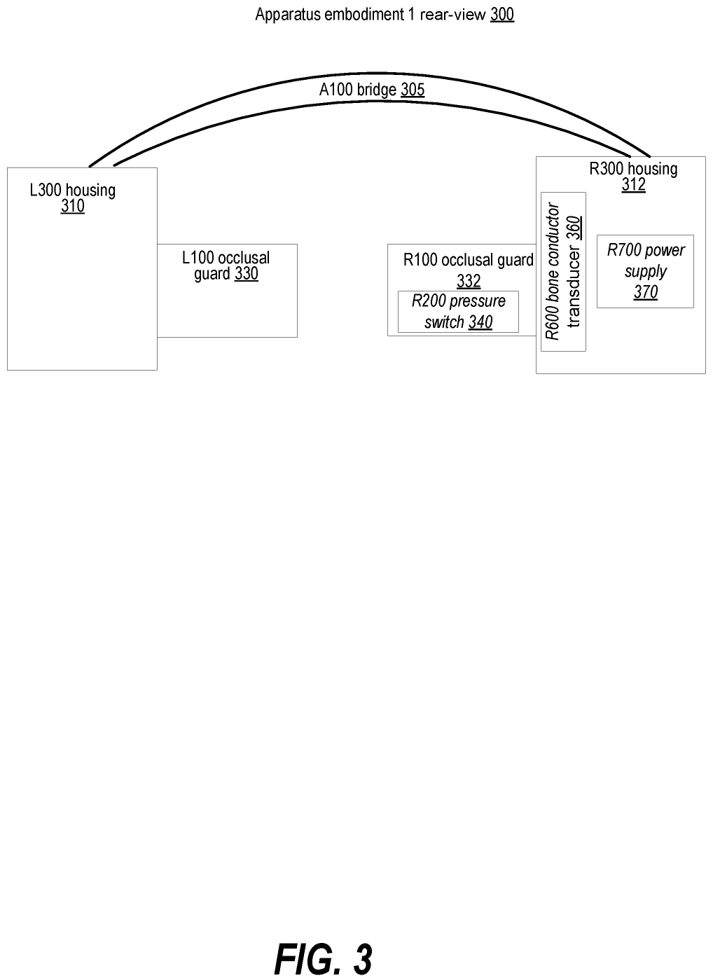

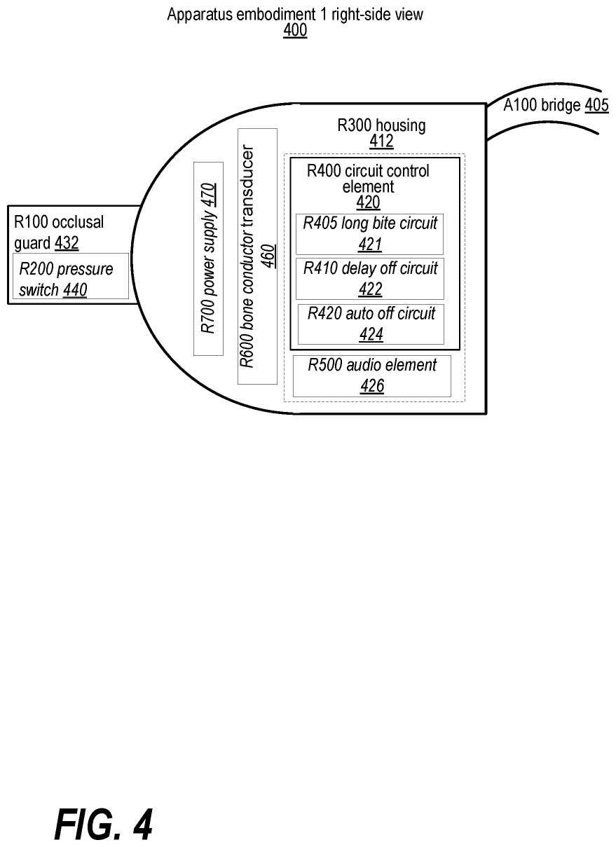

FIG. 3 and FIG. 4 display different view angles of the first embodiment to provide better understanding of how the first embodiment is used.

FIG. 3 is schematic depiction of the various components of the apparatus embodiment 1 a rear-view 300 shown in FIG. 2. An A100 bridge 305 flexible fastener connects to a L300 housing 310 for the left side of a mouth and to a R300 housing 312 for the right side of the mouth. Each of the two housings may be integrated with corresponding occlusal guards. The L300 housing 310 is integrated with a L100 occlusal guard 330. The R300 housing 312 is integrated with a R100 occlusal guard 332. The housings may be placed in an oral vestibule, the area between the teeth and the cheeks. A R200 pressure switch 340 is integrated within the R100 occlusal guard 332. The R300 housing 312 houses a R600 bone conductor transducer 360 (can be placed inside a sealed chamber, immediately adjacent to the R100 occlusal guard 332, and filled with non-electricity-conductive silicone gel), and a R700 power supply 370.

FIG. 4 is schematic depiction of the various components of the apparatus embodiment 1 right-side view 400 shown in FIG. 2. An A100 bridge 405 flexible fastener connects to a L300 housing for the left side of a mouth (not shown) and to a R300 housing 412 for the right side of the mouth. Each of the two housings may be integrated with corresponding occlusal guards. The R300 housing 412 houses a R400 circuit control element 420, a R500 audio element 426, a R600 bone conductor transducer 460, and a R700 power supply 470. The R400 circuit control element 420 may include a R405 long bite circuit 421, a R410 delay off circuit 422, and a R420 auto off circuit 424. A R200 pressure switch 440 is integrated within a R100 occlusal guard 432 and connected to the R400 circuit control element 420 in the R300 housing 412. The R600 bone conductor transducer 460 (may be placed inside a sealed chamber, immediately adjacent to the R100 occlusal guard 432, and filled with non-electricity-conductive silicone gel).

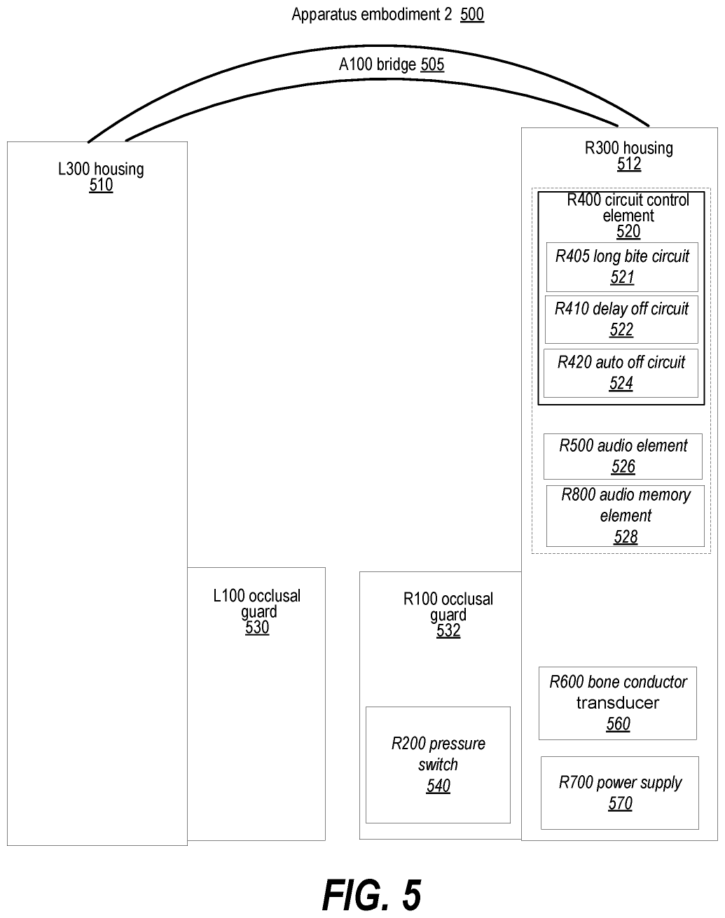

FIG. 5 is a schematic depiction including various components of a second embodiment of an apparatus supporting the operational flow in FIG. 1, the apparatus embodiment 2 500. The apparatus may be shaped like commonly available basic night guards. The second embodiment is like the first embodiment, with an additional R800 audio memory element 528, with enhanced R500 circuit control element capacity, to enable the user to upload customizable audio signals to the second embodiment to be played when this second embodiment is turned on.

An A100 bridge 505 flexible fastener connects a L300 housing 510 for the left side of a mouth to a R300 housing 512 for the right side of the mouth. Each of the two housings may be integrated with corresponding occlusal guards. The L300 housing 510 is integrated with a L100 occlusal guard 530. The R300 housing 512 is integrated with a R100 occlusal guard 532. The housings 510 and 512 may be placed in an oral vestibule, the area between the teeth and the cheeks. A R200 pressure switch 540 is integrated within the R100 occlusal guard 532 and connected to a R400 circuit control element 520 in the R300 housing 512. The R300 housing 512 houses the R400 circuit control element 520, a R500 audio element 526, the R800 audio memory element 528, a R600 bone conductor transducer 560 (may be placed inside a sealed chamber, immediately adjacent to the R100 occlusal guard 532, and filled with non-electricity-conductive silicone gel), and a R700 power supply 570. The R400 circuit control element 520 may include a R405 long bite circuit 521, a R410 delay off circuit 522, and a R420 auto off circuit 524. In this embodiment, the R800 audio memory element 528 comprises a memory area, an audio player, functions to upload and store some preferred customizable audio signals onto the memory area and sends the stored customizable audio signals to the R600 bone conductor transducer 560 to be delivered to the intraoral apparatus user. Instead of playing customizable audio signals from the R500 audio element 526, a user can choose to play the customizable audio signals stored in the R800 audio memory element 528. The R400 circuit control element 520 has power circuits, data circuits, and process controlling logics that interconnects with and controls all other electronic components in the apparatus embodiment 2 500. By uploading customizable audio signals onto the R800 audio memory element 528, the R400 circuit control element 520 will play and replay the stored customizable audio signals continuously to the user via the R600 bone conductor transducer 560. By erasing all stored customizable audio signals from the R800 audio memory element 528, the R400 circuit control element 520 will play the customizable audio signals from the R500 audio element 526 to the user via the R600 bone conductor transducer 560.

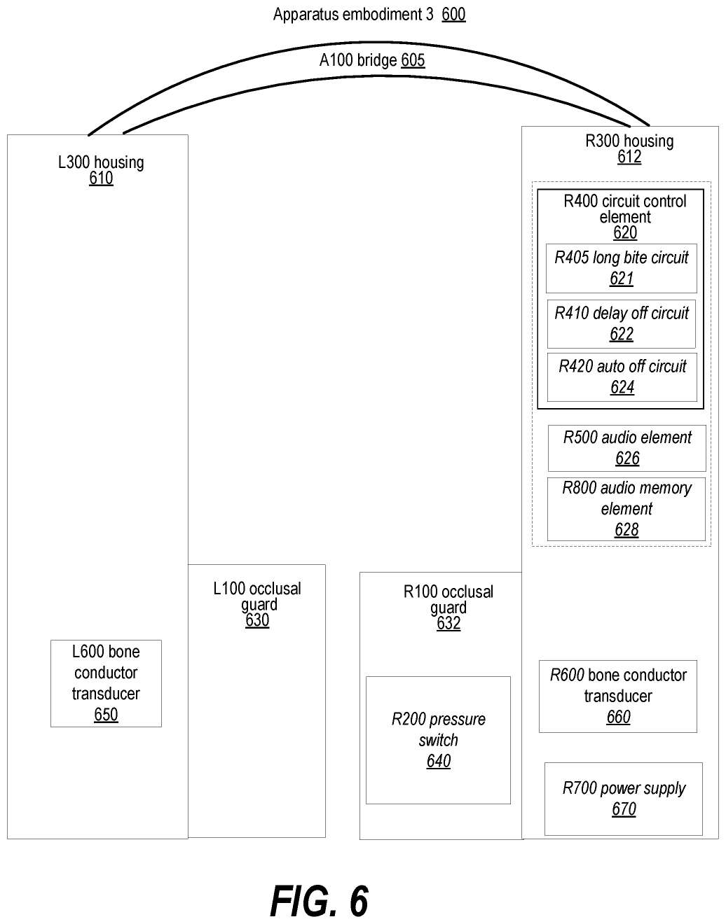

FIG. 6 is a schematic depiction including various components of a third embodiment of an apparatus supporting the operational flow in FIG. 1, the apparatus embodiment 3 600. The third embodiment is like the second embodiment, with an additional L600 bone conductor transducer 650 and an enhanced R400 circuit control element 620 with increased capacity to enable stereo effect of customizable audio signals to be delivered to both left side and right side of the user's teeth.

The apparatus may be shaped like commonly available basic night guard. An A100 bridge 605 flexible fastener connects a L300 housing 610 for the left side of a mouth to a R300 housing 612 for the right side of the mouth. Each of the two housings may be integrated with corresponding occlusal guards. The L300 housing 610 is integrated with a L100 occlusal guard 630. The R300 housing 612 is integrated with a R100 occlusal guard 632. The L300 housing 610 houses the L600 bone conductor transducer 650. The housings 610 and 612 may be placed in an oral vestibule, the area between the teeth and the cheeks. A R200 pressure switch 640 is integrated within the R100 occlusal guard 632 and connected to the R400 circuit control element 620 in the R300 housing 612. The R300 housing 612 houses the R400 circuit control element 620, a R500 audio element 626, a R600 bone conductor transducer 660 (may be placed inside a sealed chamber, immediately adjacent to the R100 occlusal guard 632, and filled with non-electricity-conductive silicone gel), a R700 power supply 670, and a R800 audio memory element 628. The R400 circuit control element 620 may include a R405 long bite circuit 621, a R410 delay off circuit 622, and a R420 auto off circuit 624. The R400 circuit control element 620 has power circuits, data circuits, and process controlling logics that interconnects with and controls all other electronic components in the apparatus embodiment 3 600. The L600 bone conductor transducer 650 can work with the R600 bone conductor transducer 660 to deliver the customizable audio signals in stereo to both sides of the user's teeth, creating stereo effect for the user's listening experience.

The possible usages of this apparatus are not limited to the three embodiments described above, as additional embodiments of this apparatus are also possible.

While embodiments have been shown and described, it will be obvious to those skilled in the art that, based upon the teachings herein, that changes, and modifications may be made without departing from this invention and its broader aspects. Therefore, the appended claims are to encompass within their scope all such changes and modifications as are within the true spirit and scope of this invention. Furthermore, it is to be understood that the invention is solely defined by the appended claims. It will be understood by those with skill in the art that if a specific number of an introduced claim element is intended, such intent will be explicitly recited in the claim, and in the absence of such recitation no such limitation is present. For non-limiting example, as an aid to understanding, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to inventions containing only one such element, even when the same claim includes the introductory phrases “one or more” or “at least one” and indefinite articles such as “a” or “an”; the same holds true for the use in the claims of definite articles.

What is claimed is:

1. A method for transmitting customizable audio signals through teeth activated by pressure comprising:
providing an intraoral apparatus with a pressure switch controlled by receiving pressure that holds a state wherein the state is one of on and off, a power source, an audio element, a control element, and a bone conduction element;
receiving by the audio element the customizable audio signals;
responsive to receiving pressure by the pressure switch when the state is off, charging the control element from the power source with a charge; and
utilizing the charge to regulate the power source to provide needed power to enable the method to transmit the customizable audio signals through the bone conduction element.

2. The method of claim 1, further comprising:
dissipating the charge when regulating the power source; and
preventing, automatically, the transmitting of the customizable audio signals when the charge is fully dissipated.

3. The method of claim 1, further comprising:
monitoring the charging of the control element for a completion of charging; and responsive to detecting the completion of charging, setting the state to off.

4. The method of claim 1, further comprising:
responsive to detecting the received pressure extended over a predetermined duration of time while the state is on, setting the state to off, and draining the charge.

5. The method of claim 1, further comprising:
responsive to detecting the received pressure extended over a predetermined duration of time while the state is off, bypassing the charging of the control element, setting the state to on, and enabling the power source to provide needed power to transmit the customizable audio signals.

6. The method of claim 1, wherein the method is used for treating bruxism and further comprises:
minimizing likelihood of waking up a user.

7. The method of claim 1, wherein the method allows a user to listen to the customizable audio signals with minimal interference from outside noise and
with minimal interference to surrounding environment.

8. An intraoral apparatus configured to transmit customizable audio signals through teeth activated by pressure comprising:
a pressure switch controlled by receiving pressure that holds a state wherein the state is one of on and off, a power source, an audio element, a control element, and a bone conduction element;
receiving by the audio element the customizable audio signals;
responsive to receiving pressure by the pressure switch when the state is off, charging the control element from the power source with a charge, and utilizing the charge to regulate the power source to provide needed power to enable the intraoral apparatus to transmit the customizable audio signals through the bone conduction element.

9. The intraoral apparatus of claim 8, further comprising:
dissipating the charge when regulating the power source; and
preventing, automatically, the transmitting of the customizable audio signals when the charge is fully dissipated.

10. The intraoral apparatus of claim 8, further comprising:
monitoring the charging of the control element for a completion of charging; and responsive to detecting the completion of charging, setting the state to off.

11. The intraoral apparatus of claim 8, further comprising:
responsive to detecting the received pressure extended over a predetermined duration of time while the state is on, setting the state to off and draining the charge.

12. The intraoral apparatus of claim 8, further comprising:
responsive to detecting the received pressure extended over a predetermined duration of time while the state is off, bypassing the charging of the control element, setting the state to on, and enabling the power source to provide needed power to transmit the customizable audio signal.

13. The intraoral apparatus of claim 8, wherein the intraoral apparatus is used for treating bruxism and minimizing likelihood of waking up a user.

14. The intraoral apparatus of claim 8, wherein the intraoral apparatus allows a user to listen to the customizable audio signals with minimal interference from outside noise and with minimal interference to surrounding environment.

15. The intraoral apparatus of claim 8, wherein the bone conduction element further comprises: a left bone conduction element and a right bone conduction element.

16. The intraoral apparatus of claim 8, further comprising:
a left occlusal guard and a right occlusal guard and wherein at least one of the left occlusal guard and the right occlusal guard is integrated with the pressure switch.

17. The intraoral apparatus of claim 16, wherein one of the left occlusal guard and the right occlusal guard is adjacent to the bone conduction element.

18. The intraoral apparatus of claim 8, wherein the bone conduction element is placed in a sealed chamber and wherein the sealed chamber is filled with non-electricity-conductive material.
